(19) United States
(12) Patent Application Publication
(10) Pub. No.: US 2023/0197207 A1
(43) Pub. Date: Jun. 22, 2023

(54) METHODS AND APPARATUS FOR MACHINE LEARNING ENHANCED INFRARED SPECTROSCOPY AND ANALYSIS

(71) Applicant: New York University, New York, NY (US)

(72) Inventors: Andrea Angulo, Brooklyn, NY (US); Lankun Yang, Staten Island, NY (US); Eray S. Aydil, New York, NY (US); Miguel A. Modestino, New York, NY (US)

(21) Appl. No.: 18/067,600

(22) Filed: Dec. 16, 2022

Related U.S. Application Data
(60) Provisional application No. 63/290,111, filed on Dec. 16, 2021.

(51) Int. Cl. G16C 20/30; G16C 20/20; G16C 20/70

(52) U.S. Cl. CPC G16C 20/30; G16C 20/20; G16C 20/70; G01N 2021/3595

(57) ABSTRACT

A method of training a machine learning model for determining the composition of a mixture includes obtaining, using Fourier-transform infrared (FTIR) spectroscopy, a spectrum for each of a plurality of mixtures its constituent components. A concentration of each constituent component is known for each of the plurality of mixtures. A plurality of features is extracted from each of the obtained spectra. A machine learning model is trained using the plurality of features. An apparatus for determining formation of a product includes a reactor for containing a reaction mixture and an FTIR spectrometer for producing a spectrum of a sample of the reaction mixture. A processor extracts features from the spectrum; provides the features to an ML model trained using a plurality of mixtures of the constituent components to obtain a concentration of one or more of the constituent components; and determines the formation of the product based on the concentration.

### CROSS REFERENCE TO RELATED APPLICATIONS

This application claims priority to U.S. Provisional Application No. 63/290,111, filed on Dec. 16, 2021, the disclosure of which is incorporated herein by reference.

### STATEMENT REGARDING FEDERALLY SPONSORED RESEARCH

This invention was made with government support under contract no. CBET-1943972 awarded by the National Science Foundation. The government has certain rights in the invention.

### BACKGROUND OF THE DISCLOSURE

Driven by an exponential increase in computational power and the ability to collect, store, and process massive amounts of data, machine learning (ML) has emerged as an invaluable tool for amplifying the performance of many technologies and businesses ranging from self-driving vehicles, targeted marketing, medical diagnostics to financial market forecasting. During the last three years, several studies implemented ML for automating and accelerating chemical process discovery, development, and optimization at the laboratory scale with impressive results, but ML has not been fully exploited in this context. Advances on this front can have an enormous impact on chemical manufacturing.

The ML approaches used for chemical process development generally rely on a feedback loop between (1) an ML-guided high-throughput experimental system featuring a chemical reactor and (2) an analytic tool to determine the compositions of the process outlet streams (FIG. 1). Within this approach, an ML algorithm selects optimal experimental conditions to test (e.g., inlet mixture composition, reactor operating conditions), which are then implemented in a reactor (e.g., thermochemical or electrochemical) by an autonomous and automated system. The outlet streams from the reactors, containing mixtures of the desired chemicals byproducts, solvents, additives, and unreacted precursors, are characterized by an analytical tool to determine their composition and the initial ML algorithm uses this information to select the next set of experiments. Determining the composition of an unknown chemical mixture is a challenging task that requires a suite of analytical tools with varying costs and speed (e.g., nuclear magnetic resonance, liquid and/or gas chromatography, mass spectrometry, and/or various optical spectroscopies, amongst others). Moreover, each technique or combination must be adapted to the chemical mixture of interest to provide complete compositional information.

### BRIEF SUMMARY OF THE DISCLOSURE

The present disclosure provides a methodology for developing and implementing machine learning (ML) models for quantitatively predicting chemical mixture compositions from their Fourier Transform infrared (FTIR) spectra. For model mixtures chosen from practical applications, linear regression (LR) and artificial neural network (ANN) models were trained with R² regression scores ranging from 0.98 to 0.99 and 0.94 to 0.98, respectively. Simpler and less computationally expensive linear regression models were consistently more accurate than ANN models, making them a superior choice for quantitative composition prediction from FTIR spectra. The present disclosure also provides discussion of the relationship between model performance and the number of spectra in the training data set and found that for both LR and ANN, regression scores increased and saturated at approximately 40 spectra for 3-component mixtures. Finally, the present disclosure shows that trained ML models (Linear Regression with PCA and Neural Networks) maintain their accuracy despite small variations in experimental conditions expected over several days. The results suggest that this methodology can enhance the analytical capabilities of FTIR spectroscopy for quantitative composition determination and find applications in inline chemical analysis applications that require fast characterization, such as autonomous chemical process development and optimization.

In an aspect, the present disclosure provides a method of training a machine learning model for determining the composition of a multicomponent mixture having known constituent components. The method includes obtaining a spectrum for each mixture of a plurality of mixtures of the constituent components. Each spectrum is produced using Fourier-transform infrared (FTIR) spectroscopy. A concentration of each constituent component is known for each mixture of the plurality of mixtures. In some embodiments, the obtained spectrum is generated by subtracting a spectrum generated using a blank sample from a spectrum generated using a sample comprising the multicomponent mixture. A plurality of features is extracted from each of the obtained spectra. For example, the plurality of features may be extracted using principal component analysis. In some embodiments, more than one spectra are obtained for each mixture of the plurality of mixtures, and the plurality of features is extracted from the more than one spectra.

A machine learning model is trained using the extracted plurality of features. The machine learning model may include, for example, a support vector regression (SVR), a ridge regression, a k-nearest neighbors (KNN), a decision tree (DT), a random forest (RF), a linear regression (LR), and/or an artificial neural network (ANN). In some embodiments, the method further includes setting an initial set of hyperparameters, evaluating a performance of the machine learning model using a test set of spectra of known mixtures, and updating the hyperparameters. The evaluating and updating steps may be repeated, for example, until an error of the machine learning model is lower than a predetermined threshold.

In another aspect, the present disclosure provides a method of determining the composition of a multicomponent mixture having known constituent components. The method includes obtaining a spectrum of the multicomponent mixture produced by scanning the mixture using FTIR spectroscopy. In some embodiments, the obtained spectrum is generated by subtracting a spectrum generated using a blank sample from a spectrum generated using a sample comprising the multicomponent mixture. A plurality of features is extracted from each of the obtained spectra. For example, the plurality of features may be extracted using principal component analysis. In some embodiments, more than one spectra are obtained for each mixture of the plurality of mixtures, and the plurality of features is extracted from the more than one spectra. The extracted plurality of features is provided to a machine learning model, which has been trained using a plurality of mixtures of the constituent components and wherein a concentration of each constituent component is known for each mixture of the plurality of mixtures used to train the machine learning model. The machine learning model may include, for example, a support vector regression (SVR), a ridge regression, a k-nearest neighbors (KNN), a decision tree (DT), a random forest (RF), a linear regression (LR), and/or an artificial neural network (ANN). The method includes obtaining a concentration of one or more constituent components of the multicomponent mixture from the trained machine learning model.

In another aspect, the present disclosure provides a method of determining formation of a product in a reaction mixture. The method includes obtaining a spectrum of the multicomponent mixture produced by scanning the mixture using FTIR spectroscopy. In some embodiments, the obtained spectrum is generated by subtracting a spectrum generated using a blank sample from a spectrum generated using a sample comprising the multicomponent mixture. A plurality of features is extracted from each of the obtained spectra. For example, the plurality of features may be extracted using principal component analysis. In some embodiments, more than one spectra are obtained for each mixture of the plurality of mixtures, and the plurality of features is extracted from the more than one spectra. The extracted plurality of features is provided to a machine learning model, which has been trained using a plurality of mixtures of the constituent components and wherein a concentration of each constituent component is known for each mixture of the plurality of mixtures used to train the machine learning model. The machine learning model may include, for example, a support vector regression (SVR), a ridge regression, a k-nearest neighbors (KNN), a decision tree (DT), a random forest (RF), a linear regression (LR), and/or an artificial neural network (ANN).

The method includes obtaining from the trained machine learning model a concentration of one or more constituent components of the reaction mixture. The steps of obtaining a spectrum of the reaction mixture, extracting a plurality of features, providing the extracted features to a machine learning model, and obtaining a concentration of one or more constituent components may be repeated until the concentration of the one or more constituent components reaches a predetermined threshold, to determine the formation of the product. In some embodiments, the method includes quenching the reaction mixture when the concentration of the one or more constituent components reaches a predetermined threshold.

In another aspect, the present disclosure provides an apparatus for determining formation of a product. The apparatus includes a reactor configured to contain the reaction mixture. An FTIR spectrometer is configured to receive a sample of the reaction mixture from the reactor and to produce a spectrum of the sample of the reaction mixture. A processor is in communication with the FTIR spectrometer. The processor is configured to extract a plurality of features from the spectrum; provide the extracted plurality of features to a machine learning model trained using a plurality of mixtures of the constituent components, wherein a concentration of each constituent component is known for each mixture of the plurality of mixtures; obtain from the trained machine learning model a concentration of one or more constituent components of the reaction mixture; and determine the formation of the product when the concentration of the one or more constituent components reaches a predetermined threshold.

In some embodiments, the apparatus further includes a flow cell in fluid communication with the reactor. The FTIR spectrometer may be configured to receive the sample by way of the flow cell. The FTIR spectrometer is configured to periodically receive a sample of the reaction mixture from the reactor and to produce a spectrum of the sample of the reaction mixture. The processor may be further configured to repeat the steps of extracting a plurality of features, providing the extracted features to a machine learning model, and obtaining a concentration of one or more constituent components for each spectrum produced by the FTIR spectrometer. In some embodiments, the processor is configured to provide a product signal when the concentration of the one or more constituent components reaches the predetermined threshold.

In another aspect, the present disclosure provides a non-transitory computer-readable medium having stored thereon a program for instructing a processor to perform any of the methods disclosed herein. For example, the stored instructions may instruct a processor to: obtain a spectrum of a reaction mixture, wherein the spectrum is produced using Fourier-transform infrared (FTIR) spectroscopy; extract a plurality of features from the spectrum; provide the extracted plurality of features to a machine learning model trained using a plurality of mixtures of the constituent components, wherein a concentration of each constituent component is known for each mixture of the plurality of mixtures; obtain from the trained machine learning model a concentration of one or more constituent components of the reaction mixture; and determine the formation of the product when the concentration of the one or more constituent components reaches a predetermined threshold, to determine formation of the product. The stored program may further include instructions to operate an FTIR spectrometer to produce the spectrum of the reaction mixture.

### DESCRIPTION OF THE DRAWINGS

For a fuller understanding of the nature and objects of the disclosure, reference should be made to the following detailed description taken in conjunction with the accompanying drawings.

FIG. 1. Diagram of autonomous process discovery, development, and optimization system composed of an ML-guided high-throughput experimental subsystem and an analytic tool to determine the compositions of the process outlet streams. In some embodiments, the present disclosure provides an ML-enhanced FTIR analytical tool for reactor outflow mixture characterization.

FIG. 2. (a-g) The FTIR absorption spectra of pure components of an experimental embodiment. (h) The spectrum resulting from a linear combination of spectra in (a-g), using a molar concentration of 0.05% for each component./

FIG. 3. The principal component analysis(PCA) explained variance, individual (bars), and cumulative (black line). Only 6 principal components were used to capture the variance in data sets with spectra from 6 chemical component mixtures.

FIG. 4. Coefficient of determination, R² averaged over the 6 components for different ML algorithms and for a base case of AN, ADN, and PN in water, applying PCA as a preprocessing step. Two hundred simulated spectra and an 80%-20% train-test partition were used in the analysis.

FIG. 5. MAE as a function of training set size for (a, b) LR and (c, d) ANN with and without PCA. These results were obtained using simulated spectra for our model 6-component mixture with noise factor (NF)=0.5. For ANN, 1 hidden layer with 12 neurons was used with the ‘relu’ activation function and a batch size of 60. When PCA was applied, only 6 PCs were included in the analysis.

FIG. 6. MAE as a function of NF for (a) LR with PCA and (b) ANN using the model 6-component mixture with Nₜᵣₐᵢₙ=400. For ANN, 1 hidden layer with 12 neurons was used with ‘relu’ activation function and a batch size of 60.

FIG. 7. MAE (averaged over all components) as a function of NF for (a) LR with PCA and (b) ANN over mixtures with different numbers of components and using Nₜᵣₐᵢₙ=400. For ANN, 1 hidden layer with 12 neurons was used with the ‘relu’ activation function and a batch size of 60.

FIG. 8. MAE (averaged over all components) as a function of NF for (a) LR with PCA and (b) ANN models applied to different chemical mixtures and using Nₜᵣₐᵢₙ=400. For ANN, 1 hidden layer with 12 neurons was used with a ‘relu’ activation function and a batch size of 60.

FIG. 9. (a) A schematic illustration of the experimental set-up with four pumps. Components are mixed using T-mixers and then delivered to a transmission flow cell, with ZnSe windows, inside the sample compartment of an FTIR spectrometer. The collected spectra were used to train and test the ML model for concentration prediction. (b) Photograph of the pumping system and FTIR spectrometer

FIG. 10. FTIR spectral measurements for glycerol (gly), isopropanol (IPA), and 1-butanol (1-but) mixtures at different mass concentrations, C_{gly}, C_{IPA}, C_{1-but}, respectively.

FIG. 11. (a-e) <MAE> [wt %] for 1-, 2- and 3-component mixtures, for ANN and LR models. Over 200 models were evaluated, each with a different train/test subset and the average MAE is reported. (f) R² for the four mixtures considered.

FIG. 12. Predicted (C_{pred}) compared to real (Cᵣₑₐₗ) mass concentrations [wt %] for LR models on (a) 1-Gly, (b) 2-AN, (c) 3-Gly, (d) 3-AN, and (e) 4-AN mixture.

FIG. 13. Model performance in terms of <R²> as a function of training set size for 3-Gly and 3-AN mixtures. For each point, 200 models were trained, and thus the average R² is reported

FIG. 14. Illustration of simulated noise introduction to spectra at three different NF levels for an aqueous solution containing AN, ADN, and PN.

FIG. 15. A flowchart for a general approach to ML model development with (a) simulated generated and (b) experimentally collected data.

FIG. 16. Weight (or loading) per component, for the five components with the highest explained variance vs. wavenumber, resulting from PCA applied to (a) 1-Gly, (b) 2-AN, (c) 3-Gly, (d) 3-AN, and (e) 4-AN mixtures.

FIG. 17. Principal component analysis explained variance, individual (blue bars), and cumulative (black line), for (a) 1-Gly, (b) 2-AN, (c) 3-Gly, (d) 3-AN, and (e) 4-AN mixtures.

FIG. 18. Regression scores R² for convolutional neural networks (CNN), for different noise to signal levels, for single component solutions of propionitrile in water. CNN configuration is: 1 convolution layer (filters=30, activation=‘relu’), 1 max pooling layer (pool_size=100, strides =10) and two hidden layers (11 and 12 neurons respectively, activation=‘relu’).

FIG. 19. A chart depicting a method according to an embodiment of the present

disclosure.

FIG. 20. A chart depicting a method according to another embodiment of the present disclosure.

FIG. 21 is a diagram of an apparatus according to another embodiment of the present disclosure.

### DETAILED DESCRIPTION OF THE DISCLOSURE

Autonomous chemical process development and optimization methods use algorithms to explore the operating parameter space based on feedback from experimentally determined exit stream compositions. Measuring the compositions of multicomponent streams is challenging, requiring multiple analytical techniques to differentiate between similar chemical components in the mixture and determine their concentration. Herein, a universal analytical methodology based on multitarget regression machine learning (ML) models is described to rapidly determine chemical mixtures' compositions from Fourier Transform Infrared (FTIR) absorption spectra. Simulated FTIR spectra for up to 6 components in water were used and seven different ML algorithms were tested to develop the methodology. All algorithms resulted in regression models with mean absolute errors (MAE) between 0-0.27 wt %. The methodology was validated with experimental data obtained on mixtures prepared using a network of programmable pumps in line with an FTIR transmission flow cell. ML models were trained using experimental data and evaluated for mixtures of up to 4-components with similar chemical structures, including alcohols (i.e., glycerol, isopropanol, and 1-butanol) and nitriles (i.e., acrylonitrile, adiponitrile, and propionitrile). Linear regression models predicted concentrations with coefficients of determination, R², between 0.955 and 0.986, while artificial neural network models showed a slightly lower accuracy, with R² between 0.854 and 0.977. These R² correspond to MAEs of 0.28-0.52 wt % for mixtures with component concentrations between 4-10 wt %. Thus, it is demonstrated herein that ML models can accurately determine the compositions of multicomponent mixtures of similar species, enhancing spectroscopic chemical quantification for use in autonomous, fast process development and optimization.

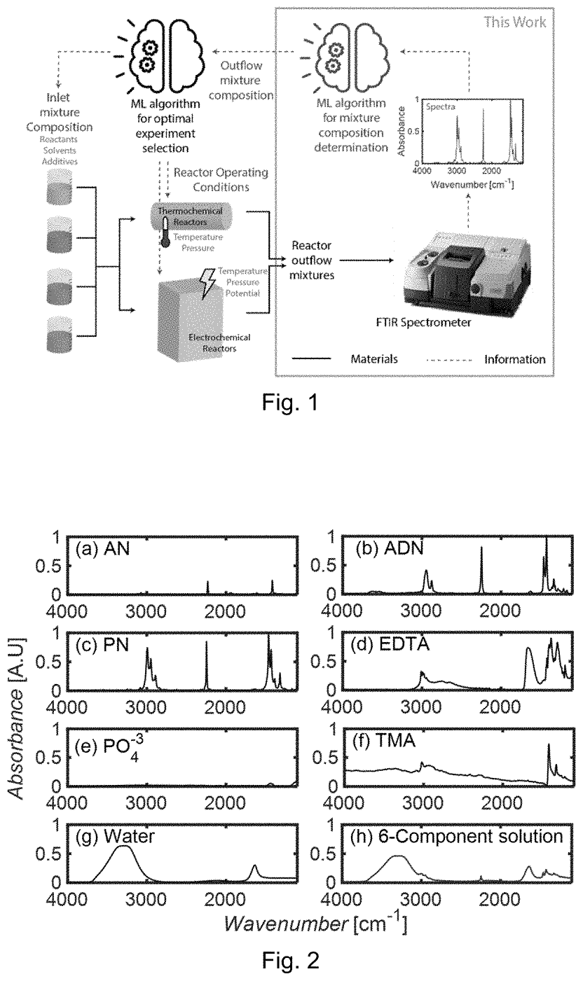

An autonomous chemical process optimization system such as that depicted in FIG. 1 would ideally use a generally applicable, non-invasive, fast, and inexpensive spectrochemical characterization tool capable of quantifying the compositions of multicomponent mixtures based on unique identifying molecular spectral features. However, interpretation of spectra collected from mixtures can be complex, and their interpretation and quantification are often challenging because of spectral feature overlap and interactions between different species. This challenge is addressed in the present disclosure by developing and demonstrating a universal ML algorithm that enables rapid inline mixture characterization using an inexpensive Fourier Transform Infrared (FTIR) spectrometer. The present approach is particularly well suited for organic synthesis and aqueous molecular solutions comprising chemicals with vibrational fingerprints, a significant fraction of cases of interest.

FTIR spectroscopy is one of the most powerful and widespread analytical techniques to determine the presence of functional groups in molecules, the compositions of chemical solutions, and to study chemical processes inline or in situ. FTIR-based methods often rely on the characterization of the position or absorbance of only a few spectroscopic features (absorption peaks) that are indicative of functional groups, while a large fraction of the spectra is ignored because overlapping features are difficult to discern, especially in the fingerprint region (i.e., ˜400-1500 cm⁻¹). Furthermore, when multiple analytes are present in the solution, absorption peaks from different molecules can overlap, and interactions between molecules can cause shifts in their positions, significantly increasing the complexity of the analysis.

Machine learning (ML) algorithms can enhance humans' ability to extract information from complex spectral data by learning the correlations between mixture compositions and absorption features. Such algorithms and FTIR data have already been used in specific food and materials applications. Previous studies have applied active learning to train classification algorithms and then use these algorithms to identify specific molecules in mixtures. A few studies have used regression algorithms to determine species concentrations. Recent examples of ML-enhanced FTIR analysis include the use of support vector machine (SVM) classifiers for rapid identification and quantification of components in artificial sweeteners with a prediction accuracy ranging between 60-94%, and the use of linear regression to determine electrolyte composition in lithium-ion batteries within an absolute error of 3-5 wt %. In the first case, the ML models were trained using only 131 absorbance points at selected wavenumbers, and the methodology included spectroscopy preprocessing methods (Savitzky-Golay, first derivative, and their combination). In the second, the ML methodology included multiple data preprocessing steps and manual selection of IR regions for specific functional groups pertaining to the species of interest. In both cases, the sample preparation was done by a lab operator.

Currently, there are multiple open-source and commercial software tools available that can facilitate the implementation of ML algorithms. These tools include MATLAB® PLS Toolbox software and Python's ScikitLearn, Keras, Tensorflow open-source library, among others.

Inspired by the successful implementation of ML in these specific applications, the present disclosure provides a universal algorithm that uses supervised ML models to determine the concentrations of chemical species in solutions via multitarget regression with minimal human intervention. A multicomponent mixture FTIR spectra was generated by linearly combining pure species spectra using the respective molar fractions of each component as weights. These simulated multicomponent spectra were then used to train ML algorithms and develop an ML methodology to determine the compositions of real chemical mixtures. Finally, the ML algorithms were validated and evaluated by comparing their predictions of the compositions of experimental mixtures from their measured FTIR spectra. The reactants and possible products of two chemical reactions were used as model mixture components: electroreduction of acrylonitrile (AN) to adiponitrile (ADN), a nylon precursor, and the valorization of glycerol into other high-value C₃ products. It was found that Artificial Neural Networks (ANN) and Linear Regression (LR) with Principal Component Analysis (PCA), also known as Principal Component Regressor (PCR), led to the most accurate predictions, with R² values ranging between 0.854-0.986 and mean absolute errors (MAE) between 0.28-0.52 wt %, depending on the number and identity of components, and ML algorithm.

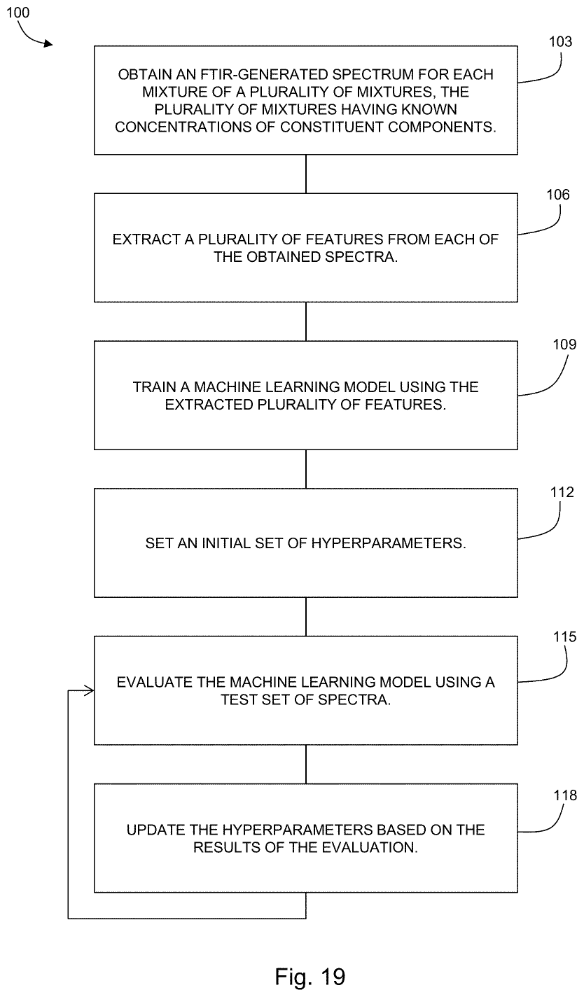

With reference to FIG. 19, the present disclosure may be embodied as a method 100 of training a machine learning model for determining the composition of a multicomponent mixture having known constituent components. The method includes obtaining 103 a spectrum for each mixture of a plurality of mixtures of the constituent components. Each spectrum is produced using FTIR spectroscopy. In each mixture of the plurality of mixtures, the concentration of each constituent component is known. As discussed below, the spectra for the plurality of mixtures may be a training set, and a test set of spectra may be used to validate the trained machine learning model. In some embodiments, more than one spectrum may be obtained for each mixture of the plurality of mixtures.

The obtained spectrum may be generated by, for example, subtracting a spectrum generated using a blank sample from a spectrum generated using a sample comprising the multicomponent mixture. In some embodiments, the blank sample does not include the constituent components of the multicomponent mixture.

In some embodiments, the obtained spectrum is subjected to post-processing. For example, post-processing may include smoothing, interpolation, peak detection, atmospheric correction, and the like, or combinations of these.

A plurality of features may be extracted 106 from each spectrum of the obtained 103 spectra. In this way, the dimensionality of each spectrum may be reduced. For example, principal component analysis may be used to extract the feature set from each spectrum. Other feature extraction techniques may be used and are within the scope of the present disclosure. In embodiments where more than one spectrum is obtained for each mixture of the plurality of mixtures, the plurality of features is extracted from the more than one spectrum.

A machine learning model is trained 109 using the extracted plurality of features. The machine learning model may be a support vector regression (SVR), a ridge regression, a k-nearest neighbors (KNN), a decision tree (DT), a random forest (RF), a linear regression (LR), or an artificial neural network (ANN) or another model.

In some embodiments, the method 100 further includes setting 112 an initial set of hyperparameters. The performance of the machine learning model i0s evaluated 115 using a test set of spectra of known mixtures. The hyperparameters may then be updated 118 based on the results of the evaluation 115. These steps may be iterated. For example, the steps may be iterated until an error of the machine learning model is lower than a predetermined threshold.

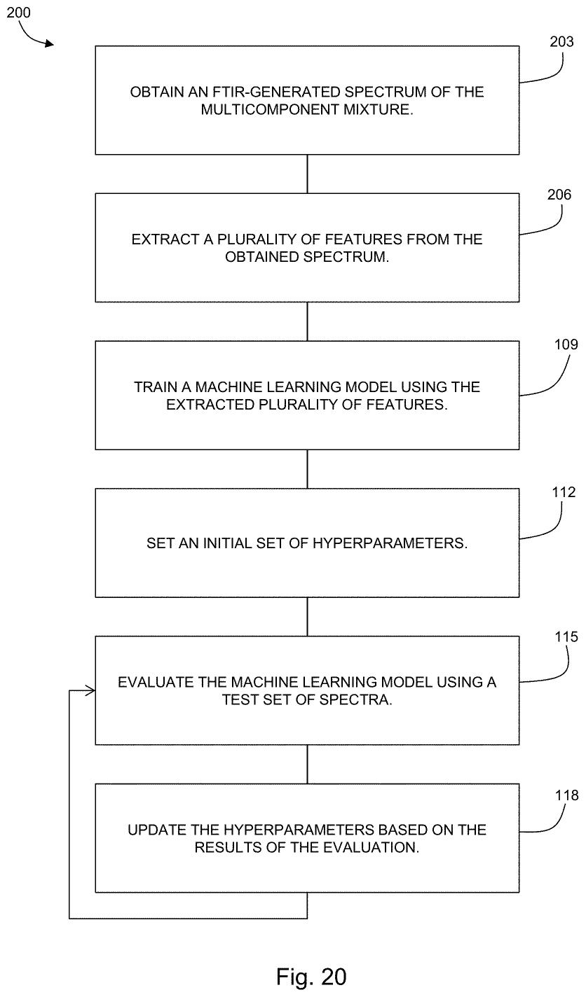

With reference to FIG. 20, in another aspect, the present disclosure may be embodied as a method 200 of determining the composition of a multicomponent mixture having known constituent components. The method 200 uses a machine learning model trained using a plurality of mixtures of the constituent components, such as, for example, the method 100 of training a machine learning model for determining the composition of a multicomponent mixture (above). The method includes obtaining 203 a spectrum of the multicomponent mixture produced by scanning the mixture using FTIR spectroscopy. In some embodiments, more than one spectra are obtained of the multicomponent mixture.

The obtained spectrum may be generated by, for example, subtracting a spectrum generated using a blank sample from a spectrum generated using a sample comprising the multicomponent mixture. In some embodiments, the blank sample does not include the constituent components of the multicomponent mixture.

In some embodiments, the obtained spectrum is subjected to post-processing. For example, post-processing may include smoothing, interpolation, peak detection, atmospheric correction, and the like, or combinations of these.

A plurality of features is extracted 206 from the obtained spectrum. In this way, the dimensionality of the spectrum may be reduced. For example, principal component analysis may be used to extract the feature set from the spectrum. Other feature extraction techniques may be used and are within the scope of the present disclosure. In embodiments where more than one spectrum is obtained of the multicomponent mixture, the plurality of features is extracted from the more than one spectrum.

The extracted 206 plurality of features is provided 209 to a machine learning model trained using a plurality of mixtures of the constituent components (trained using, for example, the method above). The trained machine learning model may be, for example, a support vector regression (SVR), a ridge regression, a k-nearest neighbors (KNN), a decision tree (DT), a random forest (RF), a linear regression (LR), or an artificial neural network (ANN), or the like. A concentration of one or more constituent components of the multicomponent mixture is obtained 212 from the trained machine learning model.

In another aspect, the present disclosure may be embodied as a method of determining formation of a product in a reaction mixture. The method includes obtaining a spectrum of the reaction mixture produced by scanning the mixture using FTIR spectroscopy. A plurality of features is extracted from the obtained spectrum. The extracted plurality of features is provided to a machine learning model trained using a plurality of mixtures of the constituent components, wherein a concentration of each constituent component is known for each mixture of the plurality of mixtures. A concentration of one or more constituent components of the reaction mixture is obtained from the trained machine learning model. The steps of obtaining a spectrum of the reaction mixture, extracting a plurality of features, providing the extracted features to a machine learning model, and obtaining a concentration of one or more constituent components are repeated, periodically, until the concentration of the one or more constituent components reaches a predetermined threshold, to determine the formation of the product.

In some embodiments, the method includes quenching the reaction mixture when the concentration of the one or more constituent components reaches a predetermined threshold.

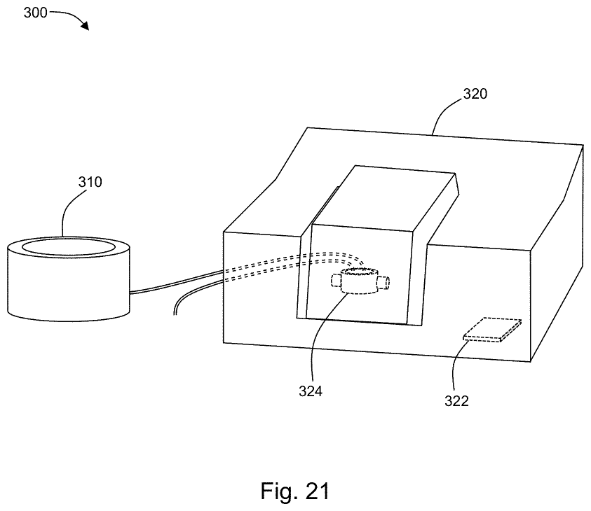

With reference to FIG. 21, in another aspect, the present disclosure may be embodied as an apparatus 300 for determining formation of a product. The apparatus 300 includes a reactor 310 configured to contain the reaction mixture and an FTIR spectrometer 320. The FTIR spectrometer is configured to receive a sample of the reaction mixture from the reactor and produce a spectrum of the sample of the reaction mixture. A processor 322 is in communication with the FTIR spectrometer 320. The processor may be configured to perform any of the methods described herein. In a particular example, the processor is configured to extract a plurality of features from the spectrum; provide the extracted plurality of features to a machine learning model trained using a plurality of mixtures of the constituent components, wherein a concentration of each constituent component is known for each mixture of the plurality of mixtures; obtain from the trained machine learning model a concentration of one or more constituent components of the reaction mixture; and determine the formation of the product when the concentration of the one or more constituent components reaches a predetermined threshold, to determine the formation of the product.

Some embodiments may include a flow cell 324 in fluid communication with the reactor 310. In such embodiments, the FTIR spectrometer 320 may be configured to receive the sample by way of the flow cell 324. The FTIR spectrometer may be configured to periodically receive a sample of the reaction mixture from the reactor and produce a spectrum of the sample of the reaction mixture. The processor may be further configured to repeat the steps of extracting a plurality of features, providing the extracted features to a machine learning model, and obtaining a concentration of one or more constituent components for each spectrum produced by the FTIR spectrometer. The processor may be further configured to provide a product signal when the concentration of the one or more constituent components reaches the predetermined threshold. For example, the processor may be configured to provide a quench signal, and the apparatus may be configured to quench the reaction mixture.

In another aspect, the present disclosure may be embodied as a non-transitory computer-readable medium encoded with computer-executable instructions, which, when executed by a processor, cause the processor to perform any of the methods described herein (such as, for example, embodiments of method 100 or method 200). For example, the stored program may comprise instructions for a processor to: obtain a spectrum of a reaction mixture, wherein the spectrum is produced using FTIR spectroscopy; extract a plurality of features from the spectrum; provide the extracted plurality of features to a machine learning model trained using a plurality of mixtures of the constituent components, wherein a concentration of each constituent component is known for each mixture of the plurality of mixtures; obtain from the trained machine learning model a concentration of one or more constituent components of the reaction mixture; and determine the formation of the product when the concentration of the one or more constituent components reaches a predetermined threshold, to determine formation of the product. The stored program may further comprise instructions to operate an FTIR spectrometer to produce the spectrum of the reaction mixture.

The term processor is intended to be interpreted broadly. For example, in some embodiments, the processor includes one or more modules and/or components. Each module/component executed by the processor can be any combination of hardware-based module/component (e.g., graphics processing unit (GPU), a field-programmable gate array (FPGA), an application-specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general-purpose processor, a central processing unit (CPU), an accelerated processing unit (APU), a field-programmable gate array (FPGA), an application-specific integrated circuit (ASIC), a digital signal processor (DSP), graphics processing unit (GPU), microprocessor, controller, microcontroller, and/or the like.

The following Statements provide various examples of the present disclosure and are not intended to be limiting.

Statement 1. A method of training a machine learning model for determining the composition of a multicomponent mixture having known constituent components, comprising: obtaining a spectrum for each mixture of a plurality of mixtures of the constituent components, wherein each spectrum is produced using Fourier-transform infrared (FTIR) spectroscopy, and wherein a concentration of each constituent component is known for each mixture of the plurality of mixtures; extracting a plurality of features from each of the obtained spectra; and training a machine learning model using the extracted plurality of features.

Statement 2. A method according to Statement 1, further comprising: setting an initial set of hyperparameters; evaluating a performance of the machine learning model using a test set of spectra of known mixtures; updating the hyperparameters; and repeating the evaluating and updating steps until an error of the machine learning model is lower than a predetermined threshold.

Statement 3. A method according to any one of the preceding Statements, wherein extracting the plurality of features comprises principal component analysis.

Statement 4. A method according to any one of the preceding Statements, wherein the machine learning model is a support vector regression (SVR), a ridge regression, a k-nearest neighbors (KNN), a decision tree (DT), a random forest (RF), a linear regression (LR), or an artificial neural network (ANN).

Statement 5. A method according to any one of the preceding Statements, wherein more than one spectra are obtained for each mixture of the plurality of mixtures, and the plurality of features is extracted from the more than one spectra.

Statement 6. A method according to any one of the preceding Statements, wherein the obtained spectrum is generated from subtracting a spectrum generated using a blank sample from a spectrum generated using a sample comprising the multicomponent mixture.

Statement 7. A method according to Statement 6, wherein the blank sample does not comprise the constituent components of the sample comprising the multicomponent mixture.

Statement 8. A method according to any one of the preceding Statements, wherein the obtained spectrum is subjected to post-processing.

Statement 9. A method according to Statement 8, wherein the post-processing comprises smoothing, interpolation, peak detection, atmospheric correction, or a combination thereof.

Statement 10. A method of determining the composition of a multicomponent mixture having known constituent components, comprising: obtaining a spectrum of the multicomponent mixture produced by scanning the mixture using FTIR spectroscopy; extracting a plurality of features from the obtained spectrum; providing the extracted plurality of features to a machine learning model trained using a plurality of mixtures of the constituent components, wherein a concentration of each constituent component is known for each mixture of the plurality of mixtures; and obtaining a concentration of one or more constituent components of the multicomponent mixture from the trained machine learning model.

Statement 11. A system according to Statement 10, wherein extracting the plurality of features comprises principal component analysis.

Statement 12. A method according to Statement 10, wherein the machine learning model is a support vector regression (SVR), a ridge regression, a k-nearest neighbors (KNN), a decision tree (DT), a random forest (RF), a linear regression (LR), or an artificial neural network (ANN).

Statement 13. A method according to Statement 10 or Statement 12, wherein more than one spectra are obtained for the multicomponent mixture.

Statement 14. A method according to any one of Statements 10, 12, or 13, wherein the obtained spectrum is generated from subtracting a spectrum generated from a blank sample from a spectrum generated from a sample comprising the multicomponent mixture.

Statement 15. A method according to Statement 14, wherein the blank sample does not comprise the constituent components of the sample comprising the multicomponent mixture.

Statement 16. A method of according to any one of Statements 10 or 12-15, wherein the obtained spectrum is subjected to post-processing.

Statement 17. The method according to Statement 16, wherein the post-processing comprises smoothing, interpolation, peak detection, atmospheric correction, or a combination thereof.

Statement 18. A method of determining formation of a product in a reaction mixture, comprising: obtaining a spectrum of the reaction mixture produced by scanning the mixture using FTIR spectroscopy; extracting a plurality of features from the obtained spectrum; providing the extracted plurality of features to a machine learning model trained using a plurality of mixtures of the constituent components, wherein a concentration of each constituent component is known for each mixture of the plurality of mixtures; obtaining from the trained machine learning model a concentration of one or more constituent components of the reaction mixture; and repeating, periodically, the steps of obtaining a spectrum of the reaction mixture, extracting a plurality of features, providing the extracted features to a machine learning model, and obtaining a concentration of one or more constituent components until the concentration of the one or more constituent components reaches a predetermined threshold, to determine the formation of the product.

Statement 19. A method according to Statement 18, further comprising quenching the reaction mixture when the concentration of the one or more constituent components reaches a predetermined threshold.

Statement 20. An apparatus for determining formation of a product, comprising: a reactor configured to contain the reaction mixture; an FTIR spectrometer configured to receive a sample of the reaction mixture from the reactor and to produce a spectrum of the sample of the reaction mixture; and a processor in communication with the FTIR spectrometer, the processor configured to: extract a plurality of features from the spectrum; provide the extracted plurality of features to a machine learning model trained using a plurality of mixtures of the constituent components, wherein a concentration of each constituent component is known for each mixture of the plurality of mixtures; obtain from the trained machine learning model a concentration of one or more constituent components of the reaction mixture; and determine the formation of the product when the concentration of the one or more constituent components reaches a predetermined threshold, to determine formation of the product.

Statement 21. An apparatus according to Statement 20, further comprising a flow cell in fluid communication with the reactor, and wherein the FTIR spectrometer is configured to receive the sample by way of the flow cell.

Statement 22. An apparatus according to Statement 20 or Statement 21, wherein the FTIR spectrometer is configured to periodically receive a sample of the reaction mixture from the reactor and to produce a spectrum of the sample of the reaction mixture.

Statement 23. An apparatus according to Statement 22, wherein the processor is further configured to repeat the steps of extracting a plurality of features, providing the extracted features to a machine learning model, and obtaining a concentration of one or more constituent components for each spectrum produced by the FTIR spectrometer.

Statement 24. An apparatus according to Statement 23, wherein the processor is configured to provide a product signal when the concentration of the one or more constituent components reaches the predetermined threshold.

### Results and Discussion

### Machine Learning Methodology Development

To develop a robust ML approach, the performance of various models was evaluated using the absorbance (A.U.) at n different wavenumbers (wn), Ā=[A₁, . . . , Aₙ], as predictor variables, and the concentrations of all (m of them) mixture components, C̅=[C₁, . . . , Cₘ] as target variables. Both n and m can vary based on the spectrometer resolution and the number of mixture components, respectively. As a non-limiting, model system, we first considered mixtures of up to 6 components with similar absorption features and relevant to the electrochemical production of nylon precursors: acrylonitrile (AN), adiponitrile (ADN), propionitrile (PN), ethylenediaminetetraacetic acid (EDTA), phosphate ions (PO₄⁻) and tetramethylammonium ions (TMA), in aqueous solutions (D. E. Blanco and M. A. Modestino, Trends in Chemistry, 2019, 1, 8-10). The individual spectra of each of these components are shown in FIG. 2. Mixture FTIR spectra were generated by linearly combining pure species spectra according to Beer's Law,

$${A}_{j} = {\sum }_{i = 1}^{m} {C}_{i} {A}_{j}^{i} \qquad ( 1 )$$

where Aⱼ is the absorbance of the multicomponent solution at the j^{th} wn, Aⱼⁱ is the absorbance of the pure species spectra at the j^{th} wn for the i^{th} component, and Cᵢ is the molar concentration of the i^{th} species. Beer's law can be used to estimate the component absorption at low concentrations when there is no significant interaction between functional groups that cause characteristic peaks to shift in the spectra. Signal-to-Noise ratio (S/N) can also be an important variable and was considered. S/N can vary depending on the acquisition speed, the light source's intensity, the sample, and spectrometer environment, and the spectrometer used. Simulated noise was introduced into the spectra as a source of non-ideality, first by randomly assigning deviations from zero to a maximum value of ±0.05 A.U. to the absorbance values at each wavenumber and then multiplying these deviation values by a noise factor, NF, that ranges from 0 (no noise introduced) to 1 (highest noise). NF was used to evaluate the performance of the ML algorithms under different amounts of noise. Hereafter, we refer to computer-generated spectra generated as described above simulated samples or simulated spectra to distinguish them from experimentally measured spectra.

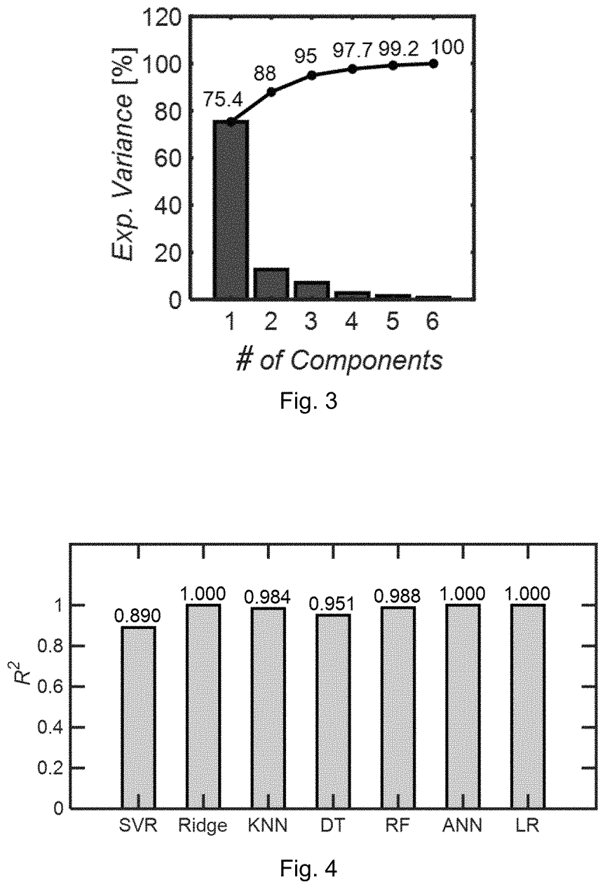

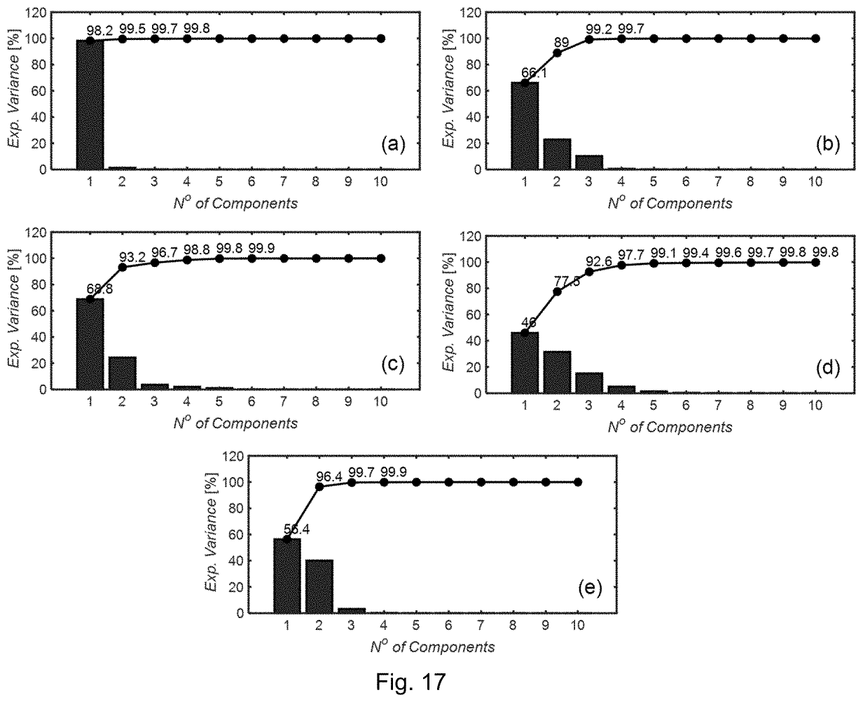

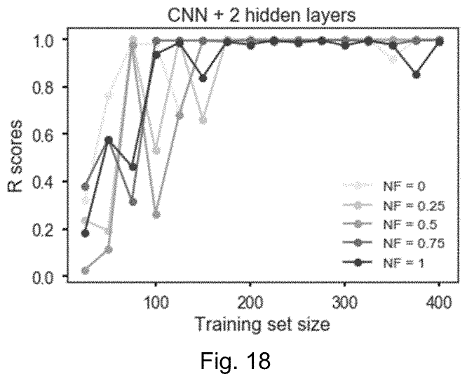

Data preprocessing: dimensionality reduction. Given the large number of predictor variables (2760 absorbance values between 4000-1000 cm⁻¹ in the model system embodiment), we implemented a principal component analysis (PCA) to reduce the dimensionality of the data set, simplifying the model and possibly enhancing its robustness. PCA is a dimensionality reduction technique that groups linearly dependent predictors and outputs a set of linearly uncorrelated principal components (PCs) that represent the directions of the data with the maximum variance. FIG. 3 shows the individual and accumulated explained variance of the PCA for our model 6-component mixture. Six principal components account for nearly all (100%) of the explained variance, consistent with the number of components in the mixtures. FIG. 17 shows the explained variance per component for the other simulated samples.

Model Selection. We considered and evaluated seven different regression models to determine the most robust and accurate ML approach. We used a base case of noise-free (NF=0) 200 simulated ternary solutions of AN, ADN, and PN in water for this evaluation. FIG. 4 shows the mean absolute error (MAE) and the coefficient of determination R² for the seven different ML algorithms, including Support Vector Regression (SVR), Ridge Regression, k-Nearest Neighbors (KNN), Decision Trees (DT), Random Forests (RF), Linear Regression (LR), and Artificial Neural Networks (ANN). Ridge Regression, ANN, and LR performed the best with MAE ˜0.00% and R² ˜1.00. LR and ANN were selected for subsequent evaluation based on their simplicity and potential ability to handle non-idealities in experimental data sets, respectively.

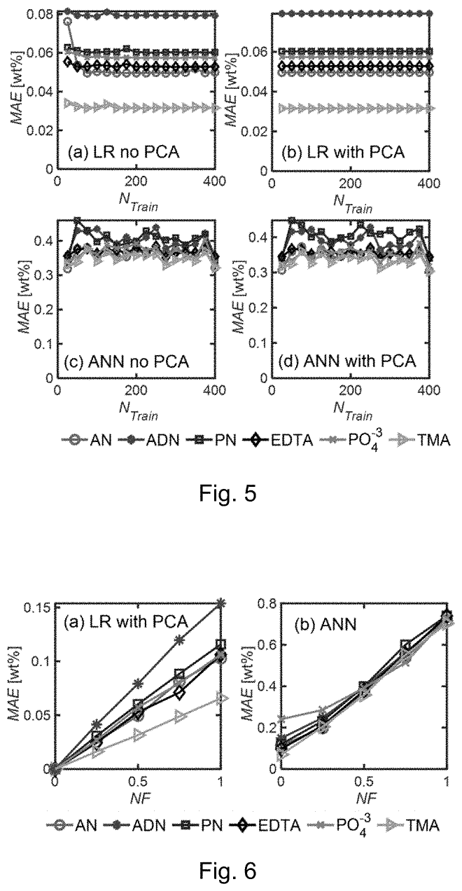

Effect of the number of training points. FIG. 5 shows the dependence of the model performance (i.e., MAE) on the number of spectra (Nₜᵣₐᵢₙ) in the training set for our model 6-component mixture. For this evaluation, NF=0.5 was chosen to simulate noise in experimental data. For the case of LR without PCA, performance stabilized for Nₜᵣₐᵢₙ≥50, while for LR with PCA, performance was nearly independent of training size for the datasets with Nₜᵣₐᵢₙ≥25. In the case of ANN, there was no clear trend between training set size and MAE, but there is higher variability between training set sizes. While the application of PCA had no noticeable effects on the performance of ANN, computational time was reduced by a factor of 10 when PCA was used.

Even in the presence of significant noise, LR performed better than ANN, with a smaller MAE between a factor of 5-10, depending on the component of interest. In LR models, TMA had the lowest MAE, which can be attributed to the substantial differences between its spectrum and other components in the range of 4000-1000 cm⁻¹, which results in a simpler differentiation. On the other hand, ADN concentration has the highest MAE given its multiple overlapping peaks with PN and AN and the lower magnitude of the peaks in the fingerprint region, which are more severely affected by noise.

Effect of simulated noise. Noise can reduce the quality of FTIR spectra and complicate analysis. Thus, it is important to determine its impact (i.e., the magnitude of NF) on the ML model prediction accuracy. FIG. 6 shows the effect of NF on MAE for the six chemicals in our model mixture. For LR with PCA and ANN, the prediction accuracy decreased for all six chemical components with increasing noise, but the MAE remained relatively low (<0.15 and 0.8 wt % for LR with PCA and ANN, respectively). For ANN, the dependence of MAE on NF did not vary significantly from component to component. On the other hand, for LR, the increase in MAE with noise was the steepest for ADN and the least steep for TMA.

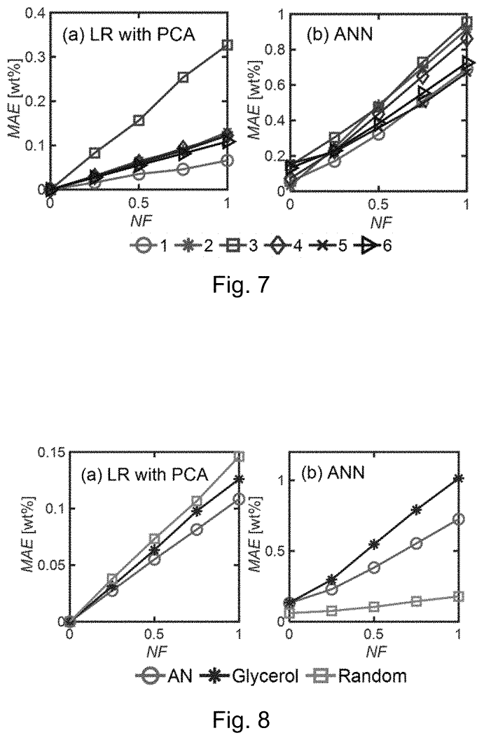

Effect of the number of chemical components. To determine the robustness of the ML methodology with numbers and identities of the chemical components, we characterized the prediction MAE (averaged over all the components in the mixture) of models trained with varying numbers of chemical components and as a function of NF (FIG. 7). Table 1 shows the components used in each one of the mixtures considered. For LR, one component (in addition to water as solvent) showed the least sensitivity to noise, while the 3-component system of AN, ADN, and PN was the most affected by noise due to the similarity of these three components. The averaged MAE is lower in 4-6 component mixtures because the errors associated with EDTA, TMA, and PO₄³⁻ are smaller than those in nitrile-containing components. In ANN models, the sensitivity to the number of components was not as pronounced, but the averaged MAEs were higher than those in LR models.

TABLE 1
Concentrations of the prepared aqueous stock solutions

| Prepared Aqueous Solution | % wt |
|---|---|
| Glycerol | 5.0-10.0 |
| IPA | 8.8 |
| 1-Butanol | 7.3 |
| AN | 4.1 |
| ADN | 4.2 |
| PN | 3.8 |

Effect of type of chemical system. We also studied if the findings from the model 6-component nitrile mixtures were transferable to mixtures containing other molecules and functional groups. To this end, we compared the nitrile-containing mixtures relevant to AN electroreduction with (i) a mixture relevant to glycerol electrooxidation, having glycerol and five possible electrooxidation products, and (ii) a mixture containing six randomly selected molecules. For the “random” case, molecules were selected from a directory containing 21 organic species spectra using random sampling. The species for these cases are shown in Table 2.

TABLE 2
Components considered for systems of different complexity
for synthetically generated data

| Number of components | Components |
|---|---|
| 1 | ADN |
| 2 | ADN, AN |
| 3 | ADN, AN, PN |
| 4 | ADN, AN, PN, EDTA |
| 5 | ADN, AN, PN, EDTA, PO₄⁻³ |
| 6 | ADN, AN, PN, EDTA, PO₄⁻³, TMA |

LR MAE as a function of NF behaved similarly for all three types of mixtures, but for ANN, the MAE of the models for the random mixture outperformed the other two, especially at high noise levels (FIG. 8). This is likely because random molecules do not necessarily have similar functional groups (fewer overlapping characteristic peaks), which makes it easier for the algorithm to differentiate between them.

### Experimental Implementation of ML Methodology

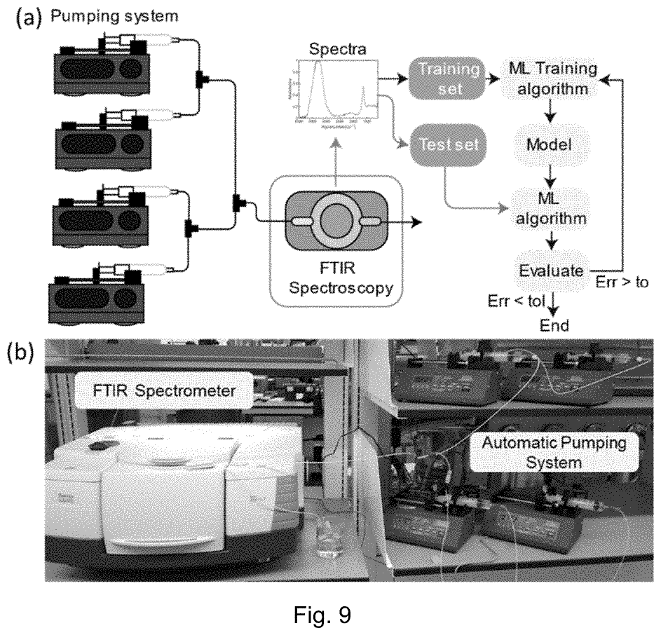

To systematically collect spectra for training the ML models, we used a network of programmable pumps that flowed solutions of selected components with known concentrations into a transmission FTIR flow cell (FIG. 9). A deionized water background was used as a reference. Based on the programmed flow rates and the spectral measurements, we collected ˜50 labelled spectra per day, which were then used to obtain LR or ANN regression models. The ML models were developed by partitioning the data randomly into training and testing sets, applying PCA and then evaluating their performance using the prediction accuracy for the test set. This process was repeated, and new hyperparameters were determined at each iteration until the error was lower than a set tolerance or the performance stopped improving. The absorbance values at each wn were used as the predictors, and mass concentrations (in wt %) were used as the predictions. The absorbance data range was limited to between 3000 and 1000 cm⁻¹ because absorption saturated outside of this range.

This methodology allowed for the collection of 50 data points per day. An operator was in charge of collecting and labelling samples and refilling the syringes with the single-component solutions once they were depleted. This methodology allowed for the autonomous collection of at least 50 data points per day, with human intervention only required to fill the syringes with single-component solutions initially. This methodology also allows us to use entire IR spectral measurement as input for our ML models without needing to select characteristic absorption regions and circumvents the problem of overlapping features of classical approaches.

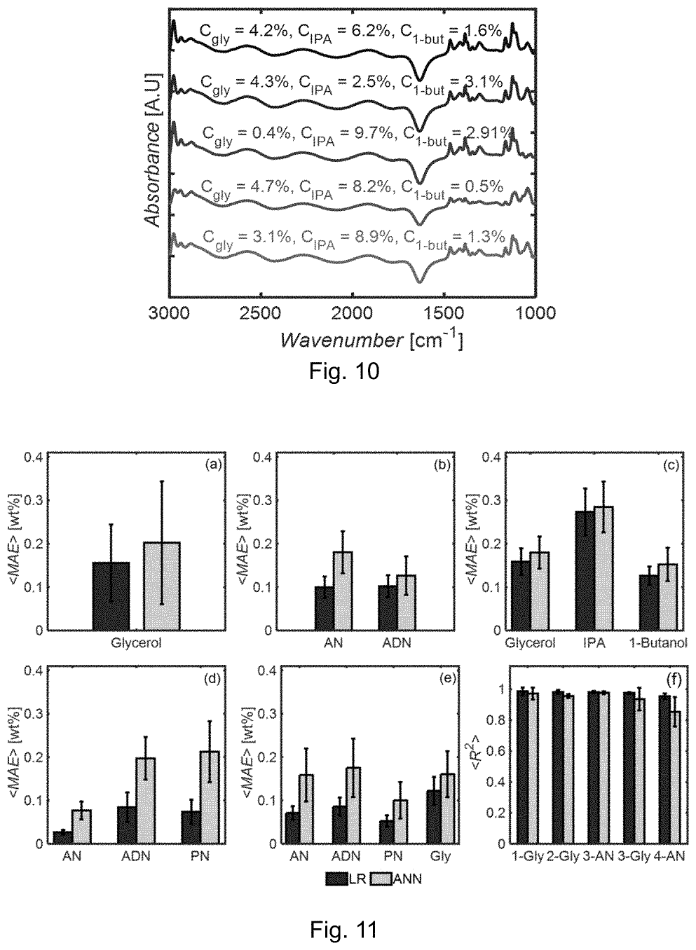

FIG. 10 shows selected FTIR spectra of 3-component mixtures with different compositions. The spectra look similar to the eye, with subtle changes in the intensities of some peaks. Without ML models here, one would have to carefully identify peaks for each species, correct for baseline, deconvolute and fit peaks, a nontrivial and arduous task to determine mixture compositions.

We show, however, that ML models with PCA can determine unknown compositions from spectra similar to these. We studied five different aqueous solutions differing in numbers and types of components in the mixture. Table 3 shows the species in the aqueous solution for each of the cases studied.

TABLE 3
Description of types of solutions studied according to
species, number of principal components for
preprocessing, and total experimental points collected

| | | Number | Experimental |
|---|---|---|---|
| Mixture | | of PC | spectra |
| label | Species | selected | collected |
| 1-Gly | Glycerol | 2 | 30 |
| 2-AN | AN, ADN | 3 | 50 |
| 3-Gly | Glycerol, IPA, 1- | 5 | 109 |
| | butanol | | |
| 3-AN | AN, ADN, PN | 5 | 67 |
| 4-AN | AN, ADN, PN, | 7 | 50 |
| | Glycerol | | |

Linear Regression and ANN Results. We implemented the LR and ANN algorithms with PCA to analyze the experimentally acquired spectra of mixtures with different compositions because these algorithms performed well when using simulated spectra. Models were trained with 80% of the spectra and then tested with the remaining 20%. We ran the training algorithm 200 times, randomly selecting different sets for training and testing. Here we report the average performance metrics, <MAE> and <R²>.

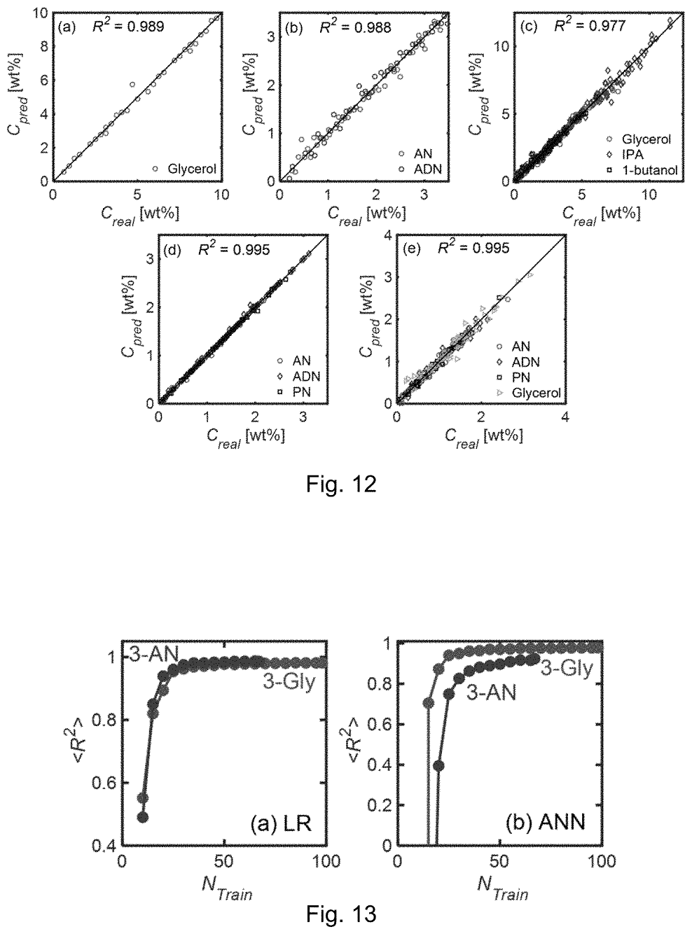

FIG. 11 compares the performances of LR implemented with PCA and ANN for the mixtures in Table 3. The <MAE> of the concentrations predicted [wt %] ranged from 0.023% to 0.28%. The <MAE> for glycerol-based mixtures did not significantly change between 1-component and 4-component mixtures. The <R²> values varied between 0.854 and 0.986, decreasing as the number of components increased. LR models had higher accuracy and weaker dependence on train/test subset combinations than ANN models for all mixtures. FIG. 12 shows the predicted and actual concentrations for the mixtures in Table 3. The subplots in this figure depict a model trained with a randomly chosen subset of the entire spectral data set, while the results in FIG. 11 show <MAE> averaged over 200 models.

Effect of number of training points. To understand the training data size requirements to produce accurate ML models, we evaluated the performance of the algorithms in terms of the <R²> for models trained with different numbers of spectra for two types of ternary aqueous solutions: an AN-based mixture (3-AN) and a Glycerol-based mixture (3-Gly). FIG. 13 shows that <R²> vs. Nₜᵣₐᵢₙ rapidly increases for these two types of mixtures but eventually saturate at ˜40 spectra. ANN is more sensitive to the training data size than LR and requires more training spectra for accurate predictions.

### Experimental Methods

Materials

Acrylonitrile (AN), adiponitrile (ADN), propionitrile (PN), 1-butanol, and glycerol were purchased from Sigma Aldrich. Isopropanol 70% was purchased from VWR. Stock solutions were prepared with deionized (DI) water.

The pumping system included two NE-1000 Programmable Syringe Pumps and two NE-4000 Programmable 2-Channel Syringe Pumps, manufactured by New Era Pump Systems: 60 ml and 30 ml BD syringes were used to load the stock solutions into the system. A Nicolet iS50 FTIR Spectrometer and OMNIC software were used for spectral data collection. The transmission flow cell was from Harrick Scientific Products and included a demountable liquid cell with Luer lock fittings and a 20 mm diameter clear aperture, equipped with a pair of 25 mm diameter ZnSe transmission windows. For all experiments, the spacing between the transmission windows was 12

### Simulated Data Generation

Simulated spectral data for mixtures of selected components were generated using Beer's law (Eq. 1). For the training set, a concentration matrix, C̅, with dimensions p×(n+1), where p is the number of points to generate, and n is the number of different components to consider, was generated according to a Sobol sequence. For the test set, a concentration matrix was created based on random distribution sampling. Compositions of individual solutes were maintained below 10% with water as a solvent. Applying a dot product between C̅ and a vertically concatenated matrix of the spectral data of the individual components Āₚᵤᵣₑ, results in a matrix of spectra, S, where each row is a new spectrum corresponding to a mixture of known concentrations.

$$[ \begin{matrix}{C}_{11} & … & … & 1 - {\sum }_{i = 1}^{n - 1} {C}_{1 i} \\ {C}_{21} & … & … & 1 - {\sum }_{i = 1}^{n - 1} {C}_{2 i} \\ ⋮ & ⋮ & ⋱ & ⋮ \\ {C}_{p 1} & {C}_{p 2} & … & 1 - {\sum }_{i = 1}^{n - 1} {C}_{pi}\end{matrix} ] \times [ \begin{matrix}{A}_{11} & {A}_{12} & … & {A}_{1 m} \\ {A}_{21} & {A}_{22} & … & {A}_{2 m} \\ ⋮ & ⋮ & ⋱ & ⋮ \\ {A}_{n 1} & {A}_{n 2} & … & {A}_{nm}\end{matrix} ] = [ \begin{matrix}{A}_{11} & {A}_{12} & … & {A}_{1 m} \\ {A}_{21} & {A}_{22} & … & {A}_{2 m} \\ ⋮ & ⋮ & ⋱ & ⋮ \\ {A}_{p 1} & {A}_{p2} & … & {A}_{pm}\end{matrix} ] \qquad ( 2 )$$

$$\overline{C} \times {\overline{A}}_{pure} = S$$

### Simulated Noise Introduction

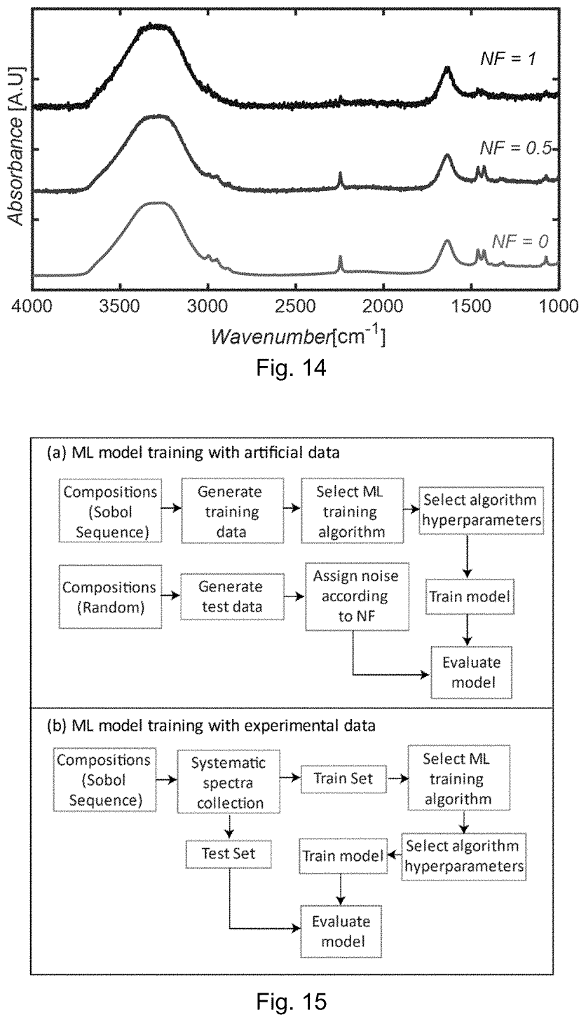

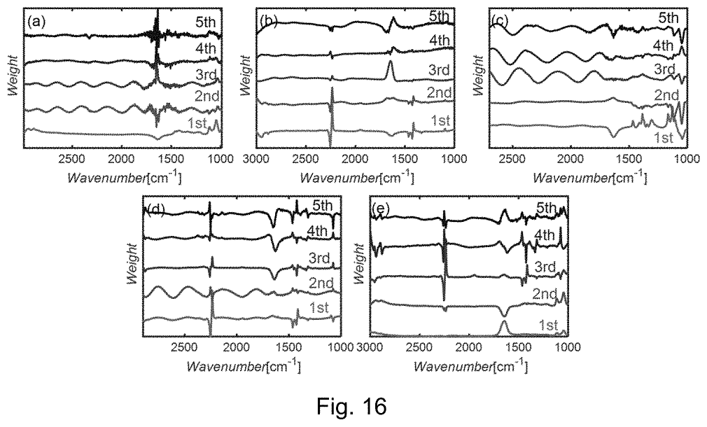

To introduce noise to the simulated test data, we defined a variable noise factor, NF, ranging from 0 (no noise assigned) to 1 (maximum noise-to-signal ratio). A number between −0.05 and +0.05 A.U. was randomly selected, multiplied by NF, and then added to each absorbance point of a spectrum. The noise range was selected based on the difference observed between the FTIR spectrum obtained from spectral libraries and the spectrum of a glycerol sample collected experimentally in our equipment using only five scans. FIG. 14 shows sample spectra at three different NF levels for an aqueous solution containing AN, ADN, and PN.

### Data Preprocessing: Principal Component Analysis

Principal component analysis (PCA) was used as a dimensionality reduction technique to decrease the number of spectral data points from thousands to up to 10 principal components for the studies conducted with simulated and experimental data. The number of principal components selected depended on the number of chemical components in the solution under study. PCA was implemented using the sklearn.preprocessing.PCA( ) function from scikit-learn, an ML library for Python.

### Machine Learning Algorithm Training and Evaluation

Machine Learning models were developed to describe relationships between solution compositions and FTIR absorbance spectra. Different ML regression algorithms available in the scikit-learn library were initially evaluated for a base case comprising 200 simulated spectra of tertiary mixtures in water, with an NF=0. The algorithms and respective scikit-learn functions are described in Table 4:

TABLE 4
Scikit-learn functions used for each ML model algorithm

| Model | Function |
|---|---|
| Linear Regression (LR) | sklearn.linear_model |
| Multilayer perceptron | sklearn.neural_network.MLPRegressor |
| regression or Artificial | |
| Neural Networks (ANN) | |
| Decision Trees | sklearn.tree.DecisionTreeRegressor |
| Random Forests (RF) | sklearn.ensemble.RandomForestRegressor |
| Support Vector Regressor | sklearn.svm.SVR |
| (SVR) | |
| Ridge Regression with Cross | sklearn.linear_model.RidgeCV |
| validation (RidgeCV) | |
| k-Nearest Neighbors (kNN) | sklearn.neighbors.KNeighborsRegressor |

Hyperparameters were optimized using sklearn.model_selection.Randomized-SearchCV. When developing regression models, the predictors or features were the absorbance values at each wavenumber, a matrix denoted S, and the target or predicted variables were the concentrations corresponding to each spectrum, contained in a concentration vector (for 1-component solution) or matrix (for a multicomponent solution) denoted C. For the experimentally collected data, S and C were divided randomly into a training and a test set, with a training/test ratio of 80%-20%. To avoid model performance dependency on the random training/test partition, each study was repeated 200 times, after which the average metrics were calculated and reported. The infrared wavenumber range for the simulated and experimental data were 4000-1000 cm⁻¹ and 3000-1000 cm⁻¹, respectively, the latter omitting the 4000-3000 cm⁻¹ range where the noise is very high due to nearly complete absorption by the water O—H stretching vibration.

FIG. 15 summarizes the general approach for developing ML regression models for the simulated and the experimentally collected data.

### FTIR Experimental Data Collection

Spectral measurements of mixtures of known concentrations were pumped into a transmission flow cell placed inside the FTIR spectrometer using a network of programmable pumps, each loaded with a single component aqueous stock solution. Concentrations of the mixture flowing through the cell were changed and controlled by varying the flow rates of the individual single-component solutions. The pumps were programmed to switch flow rates periodically at set intervals, allowing for automated spectra collection while varying compositions. For a two-component mixture, the total flow rates were maintained at 1 ml/min, 1.5 ml/min, and 2 ml/min for two-, three- and four-component mixtures, respectively. The set of compositions to sample was determined using a Sobol sequence. New sampling intervals were determined every time a new component was introduced by pumping a new solution into the flow cell and periodically taking spectral measurements until the resulting spectrum stopped changing over time. All spectra were taken with respect to the water background. Deionized water background was recorded only once at the beginning of each sampling collection session, which typically lasted for about 6 hours at the most. Datasets for one type of mixture were collected during 4 days (3-gly). Performance for the 3-gly mixtures specifically was 0.982 and 0.977 for LR and ANN, which suggests that the same model can be used for experimental campaigns that span several days without the need for recalibration.

The set of compositions to sample was determined using a Sobol sequence.

TABLE 5
Components considered for 3 different chemical systems

| Mixture Label | Components |
|---|---|
| ADN-containing | ADN, AN, PN, EDTA, PO₄⁻³, TMA |
| Glycerol-containing | Glycerol, acetic acid, dihydroxyacetone, formic |
| | acid, glycolic acid, oxalic acid |
| Random | Ethylene glycol, propionic acid, 1,2-propanediol, |
| | phenol, hexane, benzene |

TABLE 6
Hidden layers sizes and activation functions used for ANN models
(multilayer perceptron regressor) for each type of mixture considered.
The rest of the hyperparameter other than layer sizes and activation
function are the same in all cases, which are the following:
tol = 1e⁻⁵, random state = 0, solver = ‘lbfgs’,
learning rate = ‘adaptive’, batch size = 80 for
simulated data, batch size = 10 for experimental data

| | Hidden | Activation |
|---|---|---|
| Mixture | layers size | Function |
| Simulated data: | (12,) | Rectifier |
| 1 component AN | | |
| 2 components AN, ADN | | |
| 3 components AN, ADN, PN | | |
| 4 components AN, ADN, PN, EDTA | | |
| 5 components AN, ADN, PN, EDTA, PO₄⁻³ | | |
| Simulated data: | (20,) | Rectifier |
| 6 components AN, ADN, PN, EDTA, PO₄⁻³, | | |
| TMA 6 components Acetic acid, | | |
| Dihydroxyacetone, Formic acid, Glycerol, | | |
| Glycolic acid, Oxalic acid, Water | | |
| 6 components Random | | |
| Experimental data: | (2,) | Rectifier |
| 1 component Glycerol in water with | | |
| Experimental data | (10, 10) | Identity |
| 2 components AN ADN with | | |
| 3 components Glycerol, IP A, 1-butanol | | |
| 4 components AN ADN PN Glycerol | | |
| 3 components AN ADN PN | (20,) | Rectifier |

TABLE 7
Coefficient of determination R2 for different activation functions,
for ANN regression algorithms for experimental mixtures.
Under the coefficient of determination the number of
neurons per layer for each case is also noted.

| | Activation Function | | | |
|---|---|---|---|---|
| Mixture | ‘identity’ | ‘relu’ | ‘tanh’ | ‘logistic’ |
| 1-Gly | 0.9800 | 0.9808 | 0.9778 | 0.9808 |
| | (2,) | (2,) | (2,) | (2,) |
| 2-AN | 0.9810 | 0.9518 | 0.9314 | 0.9262 |
| | (10, 10) | (15, 15) | (15, 15) | (10, 10) |
| 3-Gly | 0.9815 | 0.959 | 0.9695 | 0.9712 |
| | (10, 10) | (15, 15) | (10, 10) | (10,) |
| 3-AN | 0.9215 | 0.9686 | 0.9541 | 0.7642 |
| | (15, 15) | (20,) | (20,) | (10,) |
| 4-AN | 0.8527 | 0.7768 | 0.7577 | 0.5832 |
| | (10, 10) | (20,) | (20,) | (20,) |

Although the present disclosure has been described with respect to one or more particular embodiments, it will be understood that other embodiments of the present disclosure may be made without departing from the spirit and scope of the present disclosure.

1. A method of training a machine learning model for determining the composition of a multicomponent mixture having known constituent components, comprising:
obtaining a spectrum for each mixture of a plurality of mixtures of the constituent components, wherein each spectrum is produced using Fourier-transform infrared (FTIR) spectroscopy, and wherein a concentration of each constituent component is known for each mixture of the plurality of mixtures;
extracting a plurality of features from each of the obtained spectra; and
training a machine learning model using the extracted plurality of features.

2. The method of claim 1, further comprising:
setting an initial set of hyperparameters;
evaluating a performance of the machine learning model using a test set of spectra of known mixtures;
updating the hyperparameters; and
repeating the evaluating and updating steps until an error of the machine learning model is lower than a predetermined threshold.

3. The method of claim 1, wherein extracting the plurality of features comprises principal component analysis.

4. The method of claim 1, wherein the machine learning model is a support vector regression (SVR), a ridge regression, a k-nearest neighbors (KNN), a decision tree (DT), a random forest (RF), a linear regression (LR), or an artificial neural network (ANN).

5. The method of claim 1, wherein more than one spectra are obtained for each mixture of the plurality of mixtures, and the plurality of features is extracted from the more than one spectra.

6. The method of claim 1, wherein the obtained spectrum is generated from subtracting a spectrum generated using a blank sample from a spectrum generated using a sample comprising the multicomponent mixture.

7. A method of determining the composition of a multicomponent mixture having known constituent components, comprising:
obtaining a spectrum of the multicomponent mixture produced by scanning the mixture using FTIR spectroscopy;
extracting a plurality of features from the obtained spectrum;
providing the extracted plurality of features to a machine learning model trained using a plurality of mixtures of the constituent components, wherein a concentration of each constituent component is known for each mixture of the plurality of mixtures; and
obtaining a concentration of one or more constituent components of the multicomponent mixture from the trained machine learning model.

8. The method of claim 7, wherein extracting the plurality of features comprises principal component analysis.

9. The method of claim 7, wherein the machine learning model is a support vector regression (SVR), a ridge regression, a k-nearest neighbors (KNN), a decision tree (DT), a random forest (RF), a linear regression (LR), or an artificial neural network (ANN).

10. The method of claim 7, wherein more than one spectra are obtained for the multicomponent mixture.

11. The method of claim 7, wherein the obtained spectrum is generated from subtracting a spectrum generated from a blank sample from a spectrum generated from a sample comprising the multicomponent mixture.

12. A method of determining formation of a product in a reaction mixture, comprising:
obtaining a spectrum of the reaction mixture produced by scanning the mixture using FTIR spectroscopy;
extracting a plurality of features from the obtained spectrum;
providing the extracted plurality of features to a machine learning model trained using a plurality of mixtures of the constituent components, wherein a concentration of each constituent component is known for each mixture of the plurality of mixtures;
obtaining from the trained machine learning model a concentration of one or more constituent components of the reaction mixture; and
repeating, periodically, the steps of obtaining a spectrum of the reaction mixture, extracting a plurality of features, providing the extracted features to a machine learning model, and obtaining a concentration of one or more constituent components until the concentration of the one or more constituent components reaches a predetermined threshold, to determine the formation of the product.

13. The method of claim 12, further comprising quenching the reaction mixture when the concentration of the one or more constituent components reaches a predetermined threshold.

14. An apparatus for determining formation of a product, comprising:
a reactor configured to contain the reaction mixture;
an FTIR spectrometer configured to receive a sample of the reaction mixture from the reactor and to produce a spectrum of the sample of the reaction mixture; and
a processor in communication with the FTIR spectrometer, the processor configured to:
extract a plurality of features from the spectrum;
provide the extracted plurality of features to a machine learning model trained using a plurality of mixtures of the constituent components, wherein a concentration of each constituent component is known for each mixture of the plurality of mixtures;
obtain from the trained machine learning model a concentration of one or more constituent components of the reaction mixture; and
determine the formation of the product when the concentration of the one or more constituent components reaches a predetermined threshold.

15. The apparatus of claim 14, further comprising a flow cell in fluid communication with the reactor, and wherein the FTIR spectrometer is configured to receive the sample by way of the flow cell.

16. The apparatus of claim 14, wherein the FTIR spectrometer is configured to periodically receive a sample of the reaction mixture from the reactor and to produce a spectrum of the sample of the reaction mixture.

17. The apparatus of claim 16, wherein the processor is further configured to repeat the steps of extracting a plurality of features, providing the extracted features to a machine learning model, and obtaining a concentration of one or more constituent components for each spectrum produced by the FTIR spectrometer.

18. The apparatus of claim 17, wherein the processor is configured to provide a product signal when the concentration of the one or more constituent components reaches the predetermined threshold.

19. A non-transitory computer-readable medium having stored thereon a program for instructing a processor to:
obtain a spectrum of a reaction mixture, wherein the spectrum is produced using Fourier-transform infrared (FTIR) spectroscopy;
extract a plurality of features from the spectrum;
provide the extracted plurality of features to a machine learning model trained using a plurality of mixtures of the constituent components, wherein a concentration of each constituent component is known for each mixture of the plurality of mixtures;
obtain from the trained machine learning model a concentration of one or more constituent components of the reaction mixture; and
determine the formation of the product when the concentration of the one or more constituent components reaches a predetermined threshold, to determine formation of the product.

20. The non-transitory computer-readable medium of claim 18, wherein the stored program further comprises instructions to operate an FTIR spectrometer to produce the spectrum of the reaction mixture.
